(19) United States
(12) Patent Application Publication
(10) Pub. No.: US 2024/0120069 A1
(43) Pub. Date: Apr. 11, 2024

(54) METHODS, APPARATUSES AND SYSTEMS FOR DETERMINING PROPERTY OF MEDICINE AND DEVICES AND STORAGE MEDIA

(71) Applicant: BOE Technology Group Co., Ltd., Beijing (CN)

(72) Inventor: Zhenzhong ZHANG, Beijing (CN)

(21) Appl. No.: 17/765,381

(22) PCT Filed: Apr. 29, 2021
(86) PCT No.: PCT/CN2021/090934

§ 371 Date: Mar. 30, 2022

(51) Int. Cl. G16H 20/90; G16H 50/70; G16H 70/40

(52) U.S. Cl. CPC G16H 20/90; G16H 50/70; G16H 70/40

(57) ABSTRACT

The present disclosure relates to a method, an apparatus and a system for determining a property of a medicine, and a device and a storage medium. The method includes: inputting (S101) a medical knowledge graph into a pre-trained representation network, such that the representation network outputs a representation vector of at least one node of the medical knowledge graph; and inputting (S102) a representation vector of a medicine node in the at least one node into a pre-trained determination network, such that the determination network outputs a property of a medicine corresponding to the medicine node.

### TECHNICAL FIELD

The present disclosure relates to the field of medicine property technologies, and in particular to methods, apparatuses and systems for determining a property of a medicine, and devices and storage media.

### BACKGROUND

The advance of medical treatment technologies depends on research and development of medicines and especially on research and development of Chinese herbal medicines. The properties of Chinese herbal medicines have impact on indications, combined medicines, compatibility value and the like, that is, can direct the use of Chinese herbal medicines. At present, the Chinese herbal medicines are diversified in types and research works are complex. The research on the properties of Chinese herbal medicines mainly depends on experimental research, leading to low efficiency and low accuracy of property determination.

### SUMMARY

The present disclosure provides a method, an apparatus, and a system for determining a property of a medicine, and a device and a storage medium.

According to some embodiments of the present disclosure, there is provided a method of determining a property of a medicine. The method includes:
inputting a medical knowledge graph into a pre-trained representation network, such that the representation network outputs a representation vector of at least one node of the medical knowledge graph; and
inputting a representation vector of a medicine node in the at least one node into a pre-trained determination network, such that the determination network outputs a property of a medicine corresponding to the medicine node.

In some embodiments, the representation network outputs the representation vector of the at least one node of the medical knowledge graph, including:
for each of the at least one node, obtaining an initial vector by performing initial representation for the node; and
obtaining and outputting a representation vector of the node by performing update on the initial vector at least once.

In some embodiments, performing the update on the initial vector at least once includes:
performing the update on the initial vector at least once by using one or more parent nodes and/or one or more child nodes of the node, where the one or more parent node each indicate a node pointing to the node, and the one or more child nodes each indicate a node pointed to by the node.

In some embodiments, performing the update on the initial vector at least once by using the one or more parent nodes and/or one or more child nodes of the node includes: updating the vector based on a following formula:

$${h}^{t + 1} ( {e}_{i} ) = {\sum }_{{e}_{k} ∈ Np ( {e}_{i} )} σ ( {W}_{p} \times {e}_{i} + {W}_{ph} \times {h}^{t} ( {e}_{k} ) ) + {\sum }_{{e}_{j} ∈ Nc ( {e}_{i} )} σ ( {W}_{c} \times {e}_{i} + {W}_{ch} \times {h}^{t} ( {e}_{j} ) )$$

where eᵢ represents an i-th node in N nodes of the medical knowledge graph, and i=1,.. . . . . , N, represents an activation function, Np(eᵢ) represents a set of parent nodes of eᵢ, Nc(eᵢ) represents a set of child nodes of h′(eᵢ) represents a vector obtained by performing update on the initial vector of eᵢ for t+1 times, h^{t}(eₖ) represents a vector obtained by performing update on the initial vector of eₖ for t times, h^{t}(eⱼ) represents a vector obtained by performing update on the initial vector of eⱼ for t times, where t is an integer equal to or greater than 1, and Wₚ, Wₚₕ, W_{c}, W_{ch} are network parameters of the representation network.

In some embodiments, obtaining the representation vector of the medicine node by performing the update on the initial vector at least once includes:
in response to that a time number of the update reaches a preset time number threshold, and/or, in response to that vectors after and before update are same, determining the vector obtained after the update as the representation vector of the node.

In some embodiments, the determination network outputs the property of the medicine corresponding to the medicine node, including:
based on a following formula, determining a probability that the medicine corresponding to the medicine node has the property:

$$p ( y = 1 \vert {e}_{i} ) = \frac{1}{1 + {e}^{- θ \times {h}^{n} ( {e}_{i} )}}$$

where hⁿ(eᵢ) indicates a representation vector of medicine eᵢ and θ indicates a weight vector.

In some embodiments, the method further includes:
storing the probability that the medicine has the property;
receiving medicine query information, where the medicine query information carries a medicine name and a property name;
according to the medicine query information and the stored probability that the medicine has the property, outputting a probability that a medicine corresponding to the medicine name has a property corresponding to the property name.

In some embodiments, the method further includes:
training the representation network and/or the determination network by using a plurality of nodes in a training set, where one or more medicine nodes in the plurality of nodes each are labeled with one or more true properties of a corresponding medicine.

In some embodiments, training the representation network and/or the determination network by using the plurality of nodes in the training set includes:
inputting each node in the training set into the representation network, such that the representation network outputs a representation vector of the node;
inputting a representation vector of a medicine node in the training set into the determination network, such that the determination network outputs a property of a medicine corresponding to the medicine node;
according to the output property of the medicine corresponding to the medicine node and the true property of the medicine corresponding to the medicine node, determining a network loss value; and
based on the network loss value, adjusting one or more network parameters of the representation network and/or the determination network.

In some embodiments, the method further includes:
marking labels for a plurality of medicine nodes of the medical knowledge graph, where the labels represent true properties of medicines corresponding to the medicine nodes;
determining a sub-graph formed by the plurality of medicine nodes and at least level-1 child node and parent node of each of the plurality of medicine nodes as the training set.

In some embodiments, the method further includes:
marking the property of the medicine output by the determination network for the medicine node of the medical knowledge graph.

In some embodiments, the medical knowledge graph includes one or more medicine nodes, one or more disease nodes and one or more category nodes.

In some embodiments, the property of the medicine includes anti-inflammation and non-anti-inflammation.

According to some embodiments of the present disclosure, there is provided an apparatus for determining a property of a medicine. The apparatus includes:
a representing module, configured to input a medical knowledge graph into a pre-trained representation network, such that the representation network outputs a representation vector of at least one node of the medical knowledge graph;
a determining module, configured to input a representation vector of a medicine node in the at least one node into a pre-trained determination network, such that the determination network outputs a property of a medicine corresponding to the medicine node.

In some embodiments, the representing module is specifically configured to:
for each of the at least one node, obtain an initial vector by performing initial representation for the node; and
obtain and output a representation vector of the node by performing update on the initial vector at least once.

In some embodiments, when used to perform the update on the initial vector at least once, the representing module is specifically configured to:
perform the update on the initial vector at least once by using one or more parent nodes and/or one or more child nodes of the node, where the one or more parent node each indicate a node pointing to the node, and the one or more child nodes each indicate a node pointed to by the node.

In some embodiments, when used to perform the update on the initial vector at least once by using the one or more parent nodes and/or one or more child nodes of the node, the representing module is specifically configured to:
update the vector based on a following formula:

$${h}^{t + 1} ( {e}_{i} ) = {\sum }_{{e}_{k} ∈ Np ( {e}_{i} )} σ ( {W}_{p} \times {e}_{i} + {W}_{ph} \times {h}^{t} ( {e}_{k} ) ) + {\sum }_{{e}_{j} ∈ Nc ( {e}_{i} )} σ ( {W}_{c} \times {e}_{i} + {W}_{ch} \times {h}^{t} ( {e}_{j} ) )$$

where eᵢ represents an i-th node in N nodes of the medical knowledge graph, and i=1,.. . . . . , N, represents an activation function, Np(eᵢ) represents a set of parent nodes of eᵢ, Nc(eᵢ) represents a set of child nodes of h′(eᵢ) represents a vector obtained by performing update on the initial vector of eᵢ for t+1 times, h^{t}(eₖ) represents a vector obtained by performing update on the initial vector of eₖ for t times, h^{t}(eⱼ) represents a vector obtained by performing update on the initial vector of eⱼ for t times, where t is an integer equal to or greater than 1, and Wₚ, Wₚₕ, W_{c}, W_{ch} are network parameters of the representation network.

In some embodiments, when used to obtain and output the representation vector of the node by performing the update on the initial vector at least once, the representing module is specifically configured to:
in response to that a time number of the update reaches a preset time number threshold, and/or, in response to that vectors after and before update are same, determine the vector obtained after the update as the representation vector of the node.

In some embodiments, the determining module is specifically configured to:
based on a following formula, determine a probability that the medicine corresponding to the medicine node has the property:

$$p ( y = 1 \vert {e}_{i} ) = \frac{1}{1 + {e}^{- θ \times {h}^{n} ( {e}_{i} )}}$$

where (eᵢ) indicates a representation vector of medicine eᵢ and θ indicates a weight vector.

In some embodiments, the apparatus further includes a querying module, configured to:
store the probability that the medicine has the property;
receive medicine query information, where the medicine query information carries a medicine name and a property name;
according to the medicine query information and the stored probability that the medicine has the property, output a probability that a medicine corresponding to the medicine name has a property corresponding to the property name.

In some embodiments, the apparatus further includes a training module, configured to:
train the representation network and/or the determination network by using a plurality of nodes in a training set, where one or more medicine nodes in the plurality of nodes each are labeled with one or more true properties of a corresponding medicine.

In some embodiments, the training module is specifically configured to:
input each node in the training set into the representation network, such that the representation network outputs a representation vector of the node;
input a representation vector of a medicine node in the training set into the determination network, such that the determination network outputs a property of a medicine corresponding to the medicine node;
according to the output property of the medicine corresponding to the medicine node and the true property of the medicine corresponding to the medicine node, determine a network loss value; and
based on the network loss value, adjust one or more network parameters of the representation network and/or the determination network.

In some embodiments, the apparatus further includes a training set preparing module, configured to:
mark labels for a plurality of medicine nodes of the medical knowledge graph, where the labels represent true properties of medicines corresponding to the medicine nodes;
determine a sub-graph formed by the plurality of medicine nodes and at least level-1 child node and parent node of each of the plurality of medicine nodes as the training set.

In some embodiments, the apparatus further includes a representation network, configured to:
update the medical knowledge graph according to the property of the medicine output by the determination network, and add a corresponding property attribute for the medicine node.

In some embodiments, the medical knowledge graph includes one or more medicine nodes, one or more disease nodes and one or more category nodes.

In some embodiments, the property of the medicine includes anti-inflammation and non-anti-inflammation.

According to a third aspect of embodiments of the present disclosure, there is provided a system for determining a property of a medicine. The system includes:
a representation network, configured to receive a medical knowledge graph, and output a representation vector of at least one node of the medical knowledge graph; and
a determination network, configured to receive a representation vector of a medicine node in the at least one node and output a property of a medicine corresponding to the medicine node.

According to some embodiments of the present disclosure, there is provided a system for providing medicine information. The system includes:
an inputting unit, configured to receive medicine query information of a user;
a processor, electrically connected with the inputting unit and configured to determine a property of a medicine based on the method of determining a property of a medicine according to some embodiments; and
a displaying unit, electrically connected with the processor and configured to display the property of the medicine.

According to some embodiments of the present disclosure, there is provided an electronic device, including a memory and a processor, where the memory is configured to store computer instructions executable on the processor, and the processor is configured to determine a property of a medicine according to the method of some embodiments when executing the computer instructions.

According to some embodiments of the present disclosure, there is provided a computer readable storage medium storing computer programs thereon, where the programs are executed by a processor to implement the method according to some embodiments.

It should be understood that the above general descriptions and subsequent detailed descriptions are merely illustrative and explanatory rather than limiting of the present disclosure.

### BRIEF DESCRIPTION OF THE DRAWINGS

The accompanying drawings, which are incorporated in and constitute a part of the present description, illustrate embodiments consistent with the present disclosure and serve to explain the principles of the present disclosure together with the description.

FIG. 1 is a flowchart illustrating a method of determining a property of a medicine according to some embodiments of the present disclosure.

FIG. 2 is a schematic diagram illustrating a medical knowledge graph according to some embodiments of the present disclosure.

FIG. 3 is a process diagram illustrating a method of determining a property of a medicine according to some embodiments of the present disclosure.

FIG. 4 is a structural schematic diagram illustrating an apparatus for determining a property of a medicine according to some embodiments of the present disclosure.

FIG. 5 is a structural schematic diagram illustrating a system for determining a property of a medicine according to some embodiments of the present disclosure.

FIG. 6 is a structural schematic diagram illustrating an electronic device according to some embodiments of the present disclosure.

### DETAILED DESCRIPTION OF THE EMBODIMENTS

Exemplary embodiments will be described in detail herein, with the illustrations thereof represented in the drawings. When the following descriptions involve the drawings, like numerals in different drawings refer to like or similar elements unless otherwise indicated. The embodiments described in the following examples do not represent all embodiments consistent with the present disclosure. Rather, they are merely examples of apparatuses and methods consistent with some aspects of the present disclosure as detailed in the appended claims.

The terms used in the present disclosure are for the purpose of describing particular examples only, and are not intended to limit the present disclosure. Terms determined by “a”, “the” and “said” in their singular forms in the present disclosure and the appended claims are also intended to include plurality, unless clearly indicated otherwise in the context. It should also be understood that the term “and/or” as used herein refers to and includes any and all possible combinations of one or more of the associated listed items.

It is to be understood that, although the terms “first,” “second,” “third,” and the like may be used in the present disclosure to describe various information, such information should not be limited to these terms. These terms are only used to distinguish one category of information from another. For example, without departing from the scope of the present disclosure, first information may be referred as second information; and similarly, the second information may also be referred as the first information. Depending on the context, the term “if” as used herein may be interpreted as “when” or “upon” or “in response to determining”.

The prescriptions of Chinese herbal medicines have complex compositions, and different doctors may give different prescriptions. With a prescription of a Chinese herbal medicine for treatment of lung cancer as an example, dozens of prescriptions of Chinese herbal medicines may be found in literatures, for example, a prescription formed by Beisha ginseng/Radix Glehniae, Radix Ophiopogonis, Herba Hedyotis, Pseudobulbus Cremastrae seu Pleiones, Radix Rnunculi Ternati, fried Pollen Typhae; for another example, a prescription formed by ginseng, Radix Astragali seu Hedysari, Herba Hedyotis, Radix Rnunculi Ternati, Saffron, Panax pseudo-ginseng, musk, Chinese lobelia, Curcumae Rhizoma, trumpet creeper, Cyperus rotundus, peach kernel, Akebia Fruit, gecko, Cordyceps Sinensis, sporoderm-broken spore powder of Ganoderma lucidu; for another example, a prescription formed by Pseudobulbus Cremastrae seu Pleiones, Radix Astragali seu Hedysari, green tea, propolis, sea-buckthorn, centipede, pheretima, Thunberg Fritillary Bulb, green onion, calamus, Angelica sinensis, and Golden Larch Bark; for another example, American ginseng, Ganoderma, Solanum nigrum, salvia chinensis, Pseudobulbus Cremastrae seu Pleiones, Coffee Senna, Shepaole/Rubus parviflolius L., Indian mock strawberry, Cordyceps Sinensis, and Radix Glycyrrhizae; for another example, a prescription formed by Medcinal Evodia Fruit, Marsdeniae Tenacissimae Caulis, solanum lyratum thunb, Celastrus orbiculatus Thunb, Chinese Fevervine Herb, Spatholobi Caulis, Caulis Lonicerae, Vitis quinquangularis Rehd., Sargentgloryvine Stem, Sophorae Flavescentis Radix, Rhizoma Paridis and Radix Glycyrrhizae. As seen from the above prescriptions, each prescription for treatment of lung cancer contains 6-15 ingredients. If whether each Chinese herbal medicine (e.g. Herba Hedyotis) has a certain property (e.g. anti-inflammatory) is determined through experiment, a large amount of time and labor will be required, resulting in high costs.

In terms of this, some embodiments of the present disclosure provide a method of determining a property of a medicine. As shown in FIG. 1, the flow of the determination method may include steps S101 to S102.

The determination method may be used for a Chinese herbal medicine or a western medicine, and the property determined herein may be one or more properties, such as anti-inflammation, dampness removal, Qi strengthening and the like. A result of determination for a property of a medicine by the determination method may be used to direct the use of the medicine independently, or may be combined with experimental research to direct the use of the medicine.

Furthermore, the method may be performed by an electronic device such as a terminal device, or a server. The terminal device may be user equipment (UE), mobile device, user terminal, terminal, cellular phone, cordless phone, personal digital assistant (PDA), handheld device, computing device, vehicle-mounted device, wearable device, and the like. The method may be implemented by invoking computer readable instructions stored in a memory through a processor. In another embodiment, the method may be implemented by a server which may be a local server or a cloud server or the like.

At step S101, a medical knowledge graph is input into a pre-trained representation network, such that the representation network outputs a representation vector of at least one node of the medical knowledge graph.

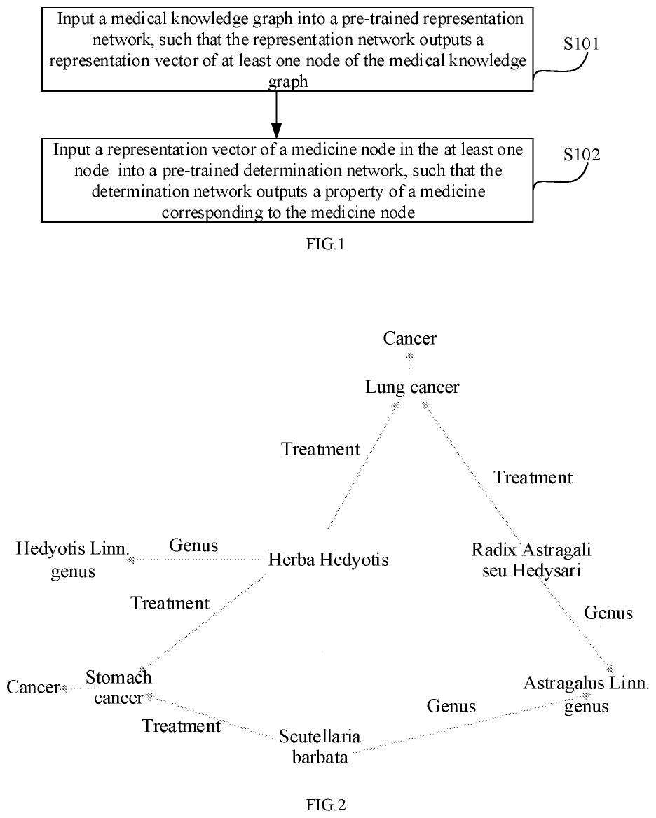

The medical knowledge graph may be a Chinese medicine knowledge graph or a medical knowledge graph of another type, which is not limited herein. The medical knowledge graph represents medical knowledge using nodes and borders, for example, an adaptive relationship between a medicine and a disease, and a type, a subordinate relationship, an attribute relationship and the like of a medicine. In some examples, the medical knowledge graph includes nodes and borders each connecting two nodes, for example, the border may have a direction (e.g. a direction of the border is represented by an arrow). In some embodiments, the nodes may include one or more medicine nodes each representing a medicine, one or more disease nodes each representing a disease, and one or more category nodes each representing a category (e.g. a category/genus of a medicine or a category/class of a disease) and the like; and the borders may include one or more treatment borders each between a medicine node and a disease node, and the border of this category points to a disease node from a medicine node, which represents that the medicine at one end is suitable for treating the disease at the other end. Further, the borders may also include one or more subordinate borders each between a medicine node and a category node, and the border of this category points to a category node from a medicine node, which represents that the medicine at one end belongs to the category at the other end. Furthermore, the borders may include one or more subordinate borders each between a disease node and a category node, and the border of this category points to a category node from a disease node, which represents that the disease at one end belongs to the category at the other end. For example, the Chinese medicine knowledge graph shown in FIG. 2 includes three medicine nodes, Herba Hedyotis, Radix Astragali seu Hedysari and Scutellaria barbata, further includes two medicine genus nodes, Astragalus Linn. genus and Hedyotis Linn. genus, further includes two disease nodes, lung cancer and stomach cancer, further includes a disease category node, cancer; and includes a treatment border that Herba Hedyotis points to lung cancer, a treatment border that Herba Hedyotis points to stomach cancer, a treatment border that Radix Astragali seu Hedysari points to lung cancer, a treatment border that Scutellaria barbata points to stomach cancer, a subordinate border that Herba Hedyotis points to Hedyotis Linn. genus, a subordinate border that Radix Astragali seu Hedysari points to Astragalus Linn. genus, a subordinate border that Scutellaria barbata points to Astragalus Linn. genus, a subordinate border that stomach cancer points to cancer, and a subordinate border that lung cancer points to cancer.

In some embodiments, a complete medical knowledge graph may be input into the pre-trained representation network, or a part of the complete medical knowledge graph, i.e. a sub-graph formed by some nodes and borders in the complete medical knowledge graph, may be input into the pre-trained representation network.

The representation network may be a neural network, for example, a graph neural network, a convolutional neural network and the like. The representation network may be pre-trained for its parameters, such that the representation network can receive a medical knowledge graph and output a representation vector of at least one node in the medical knowledge graph. The generation process of the representation vector incorporates a relationship between the node itself and other nodes, i.e. incorporates information of the node itself, other nodes, and borders between the node itself and other nodes, and therefore, the representation vector represents knowledge information about the node in the medical knowledge graph. The representation network may output the representation vectors of all nodes in the medical knowledge graph, or output the representation vectors of some nodes in the medical knowledge graph.

In addition, a dimension of the representation vector may be preset. The higher the dimension of the representation vector is, the more accurate the representation for the knowledge information of the node is, and the higher the energy consumption is. The lower the dimension of the representation vector is, the less accurate the representation for the knowledge information of the node is, and the lower the energy consumption is. For example, the dimension of the representation vector may be set to 256 dimensions, such that the accuracy that the representation vector represents the knowledge information of the node can be guaranteed and excess energy consumption can be avoided.

At step S102, a representation vector of a medicine node in the at least one node is input into a pre-trained determination network, such that the determination network outputs a property of a medicine corresponding to the medicine node.

One or more medicine nodes in the at least one node may be screened out by identifying each node in the at least one node. The determination network may be a classifier, for example, a Softmax classifier, which can determine a property of a corresponding medicine based on the representation vector. For example, the determination network can determine that a medicine has a particular property or does not have a particular property, where the property may be anti-inflammation and the like. The determination network and the representation network may be implemented by different models, or taken as different components of one model. For example, the determination network and the representation network may also form a generative adversarial network.

As can be known from the above embodiments, a medical knowledge graph is input into a pre-trained representation network, such that the representation network outputs a representation vector of at least one node in the medical knowledge graph; and a representation vector of a medicine node in the at least one node is input into a pre-trained determination network, such that the determination network outputs a property of a medicine corresponding to the medicine node. The representation network and the determination network can process the medical knowledge graph automatically to obtain the properties of the medicines in a batch, thus avoiding a low efficiency and a low accuracy resulting from experimental research on the properties of the medicines, and improving an efficiency and an accuracy of medicine research.

In some embodiments of the present disclosure, the representation network may output the representation vector of at least one node of the medical knowledge graph in the following manner: firstly, for each of the at least one node, obtaining an initial vector by performing initial representation for the node; next, obtaining and outputting the representation vector of the node by performing update on the initial vector at least once.

The initial vector of each node in the medical knowledge graph may be initialized randomly. For example, the medical knowledge graph includes a total of N nodes {eᵢ, i=1, . . . , N} and a total of M borders {r j, j=1, . . . , M}, and the initial vector of the node may be denoted as h⁰(eᵢ), i=1, . . . , N.

When update is performed on the initial vector at least once, update of at least once may be performed on the initial vector by using one or more parent nodes and/or one or more child nodes of the node. The one or more parent nodes each indicate a node pointing to the node. For example, in the medical knowledge graph shown in FIG. 2, the parent node of the Hedyotis Linn. genus is Herba Hedyotis, the parent nodes of the stomach cancer are Herba Hedyotis and Scutellaria barbata, and the parent nodes of the lung cancer are Herba Hedyotis and Radix Astragali seu Hedysari. The one or more child nodes each indicate a node pointed to by the node. For example, in the medical knowledge graph shown in FIG. 2, the child nodes of Herba Hedyotis are Hedyotis Linn. genus, lung cancer and stomach cancer, and the child nodes of the Scutellaria barbata are stomach cancer and Astragalus Linn. genus.

In some embodiments, the vector of the node eᵢ may be updated based on the following formula:

$${h}^{t + 1} ( {e}_{i} ) = {\sum }_{{e}_{k} ∈ Np ( {e}_{i} )} σ ( {W}_{p} \times {e}_{i} + {W}_{ph} \times {h}^{t} ( {e}_{k} ) ) + {\sum }_{{e}_{j} ∈ Nc ( {e}_{i} )} σ ( {W}_{c} \times {e}_{i} + {W}_{ch} \times {h}^{t} ( {e}_{j} ) )$$

where σ represents a relu activation function, Np(eᵢ) represents a set of parent nodes of the node Nc(eᵢ) represents a set of child nodes of the node and Wₚ, Wₚₕ, W_{c}, W_{ch} are parameters of a representation network.

It is noted that, e k may transverse each parent node in the set of parent nodes, and eⱼ may transverse each child node in the set of child nodes. When one node only has a parent node but a child node, a part about the child node in the above formula may be omitted; when one node only has a child node but a parent node, a part about the parent node in the above formula may be omitted.

When update of at least once is performed on the initial vector, in response to that a time number of the update reaches a preset time number threshold, and/or, in response to that vectors before and after update are same, the vector obtained after the update may be determined as the representation vector of the node. The “same” herein means completely or approximately same, and the “approximately same” means a difference between vectors before and after update is smaller than a given threshold, or a similarity between vectors before and after update is greater than a given threshold. That is, based on at least one of two conditions, the update for the representation vector is stopped. The first condition is a time number of update, where a time number threshold may be set to, for example, 9. When a vector representation of a node obtained through 9 updates is h⁹(eᵢ), i=1, . . . , N, the update is stopped and h⁹(eᵢ), i=1, . . . , N is determined as a representation vector of the corresponding node. The second condition is a change resulting from update. When the vectors before and after update are same, that is, the update no longer causes any change to the vector, the update is stopped, and the vector obtained after the update is determined as a representation vector of the corresponding node.

In this embodiment, through initial representation and further update for the vector, a final representation vector is obtained, and the representation vector can be continuously optimized using one or more parameters in the vector representation and one or more parameters in the update formula, such that the vector representation can be made close to a feature of the corresponding node as possible.

Based on the above generation manner of the representation vector, whether the medicine eᵢ has a specific property, for example, anti-inflammation, may be predicted using a Softmax classifier. For example, based on the following formula, a probability p(y=1|eᵢ) that the medicine eᵢ has the specific property may be predicted:

$$p ( y = 1 \vert {e}_{i} ) = \frac{1}{1 + {e}^{- θ \times {h}^{n} ( {e}_{i} )}}$$

where hⁿ(eᵢ) is a representation vector of the medicine eᵢ, and θ is a weight vector same as the representation vector in dimension, for example, the dimensions of the representation vector and the weight vector are both 256. For the part θ×hⁿ(eᵢ) in the formula, when the weight vector is a row vector and the representation vector is a column vector, the weight vector and the representation vector are directly multiplied; when the weight vector is a column vector and the representation vector is a column vector, the weight vector is transposed and then multiplied by the representation vector; when the weight vector is a row vector and the representation vector is a row vector, the weight vector is multiplied by a transposition result of the representation vector; when the weight vector is a column vector and the representation vector is a row vector, the weight vector is transposed and then multiplied by a transposition result of the representation vector.

After the determination network outputs the property of the medicine corresponding to the medicine node, the medical knowledge graph may be further updated based on the property of the medicine output by the determination network, and a corresponding property attribute is added for the corresponding medicine node. In some embodiments, the property of the medicine output by the determination network may be marked to the corresponding medicine node of the medical knowledge graph. For example, a probability that the medicine has a property is marked to the corresponding medicine node of the medical knowledge graph. In some embodiments, if the property node is already present in the knowledge graph, a border between the property node and the corresponding medicine node is added; if the property node is not present in the knowledge graph, the property node is added and a border between the property node and the corresponding medicine node is added.

Furthermore, after the determination network outputs the property of the medicine corresponding to the medicine node, the property of the medicine output by the determination network may be further output for a user to view. For example, a probability that the medicine has a property is output for a user to view.

When the above probability is greater than a given threshold, it is determined that the medicine eᵢ has the specific property, for example, anti-inflammation. The threshold may be set according to requirements, for example, may be set to 50%, which is not limited herein.

In some embodiments, a probability that each medicine has the specific property is output or stored, for example, may be stored in a terminal device or a server.

In some embodiments, when a user inputs a name of a medicine, a stored probability that the medicine has the specific property may be queried, and the probability that the medicine has the specific property may be output. For example, after the user inputs a name of a to-be-queried medicine through a terminal, a probability, which is stored in the terminal or the server, that the medicine has one or more specific properties may be retrieved, and the probability that the medicine has one or more specific properties may be output. For example, the user may input a name of a specific medicine and a specific property of the medicine to be queried at the same time, and the probability that the specific medicine has the specific property is output.

For example, in some embodiments, the user may be any user. For example, the user may be any registered user of the above terminal or an application program of the above terminal. In some embodiments of the present disclosure, the method of determining a property of a medicine further includes: by using a plurality of nodes in a training set, training the representation network and/or the determination network, where one or more medicine nodes in the plurality of nodes are labeled with a true property of a corresponding medicine.

By training the representation network and/or the determination network using the training set, it is desired that the property of the medicine corresponding to the medicine node output by the determination network is the true property of the medicine. During a training process, the output property of the medicine gradually approximates to the true property of the medicine.

In some embodiments, the representation network and/or the determination network is trained in the following manner: firstly, each node in the training set is input into the representation network, such that the representation network outputs a representation vector of the node; next, a representation vector of a medicine node in the training set is input into the determination network, such that the determination network outputs a property of a medicine corresponding to the medicine node; next, according to the output property of the medicine corresponding to the medicine node and the true property of the medicine corresponding to the medicine node, a network loss value is determined; finally, based on the network loss value, one or more network parameters of the representation network and/or the determination network are adjusted.

In a training process, the process in which the representation network generates a representation vector and the process in which the determination network outputs a property of a medicine are same as the processing procedures performed by the trained representation network and the trained determination network in the above embodiments.

The output property of the medicine is a prediction value of the representation network and the determination network, the true property of the medicine is a true value, and the network loss value may be determined by comparing the prediction value and the true value. For example, when the medicine eᵢ has a specific property, a difference between a probability p (y=1|eᵢ) that the medicine eᵢ has the specific property and 1 may be determined as the network loss value. When the medicine eᵢ does not have a specific property, a difference between the probability p(y=1|eᵢ) that the medicine eᵢ has the specific property and θ is determined as the network loss value.

Since the network loss value can reflect a deviation of the one or more network parameters of the representation network and/or the determination network, the network loss value can be gradually minimized by adjusting the one or more network parameters of the representation network and/or the determination network, such that the difference between the probability p(y=1|eᵢ) that the medicine eᵢ has the specific property and the true probability is gradually reduced until a preset requirement is reached. At this time, adjustment to the one or more network parameters is stopped. In an example, when the network loss value is smaller than a preset loss value threshold, adjustment to the one or more network parameters of the representation network and/or the determination network is stopped; and/or, when a number of times of adjustment exceeds a preset number-of-times threshold, adjustment to the one or more network parameters of the representation network and/or the determination network is stopped. When the network loss value is smaller than the preset loss value threshold, an accuracy of a target function reaches a preset requirement; when the number of times of adjustment exceeds the preset number-of-times threshold, a maximum iteration number is reached. Therefore, in either of the two cases, the training may be ended, and a system model formed by the trained representation network and the trained determination network is stored.

In an example, the training set includes the following nodes {(eᵢ, yᵢ), i=1, . . . , K}, where yᵢ=1 represents that eᵢ has anti-inflammation, and yᵢ=0 represents that eᵢ does not have anti-inflammation. By maximizing the following target function based on stochastic gradient descent, the one or more network parameters of the representation network and/or the determination network may be adjusted.

$${\sum }_{i = 1}^{K} {y}_{i} log p ( 1 \vert {e}_{i} ) + ( 1 - {y}_{i} ) log ( 1 - p ( 1 \vert {e}_{i} ) )$$

where p(1|eᵢ) is calculated based on

$$p ( y = 1 \vert {e}_{i} ) = \frac{1}{1 + {e}^{- θ \times {h}^{n} ( {e}_{i} )}} ,$$

and hⁿ(eᵢ) is calculated based on

$${h}^{t + 1} ( {e}_{i} ) = {\sum }_{{e}_{k} ∈ Np ( {e}_{i} )} σ ( {W}_{p} \times {e}_{i} + {W}_{ph} \times {h}^{t} ( {e}_{k} ) ) + {\sum }_{{e}_{j} ∈ Nc ( {e}_{i} )} σ ( {W}_{c} \times {e}_{i} + {W}_{ch} \times {h}^{t} ( {e}_{j} ) ) ,$$

that is, the network parameters adjusted are parameters such as Wₚ, Wₚₕ, W_{c}, W_{ch}, θ and one or more parameters in σ function and one or more parameters in the representation vector.

It is noted that the specific details of the above formulae have been elaborated in the above embodiments and thus will not be repeated herein.

In some embodiments of the present disclosure, the method of determining a property of a medicine further includes a process of preparing a training set: firstly, marking labels for a plurality of medicine nodes of the medical knowledge graph, where the labels represent true properties of medicines corresponding to the medicine nodes; next, determining a sub-graph formed by the plurality of medicine nodes and at least level-1 child node and parent node of each of the plurality of medicine nodes as a training set.

Label marking may be performed for medicines having a definite medicinal property, for example, for medicines which are determined to have a particular property through experimental research, or for medicines which have been used clinically for long based on the fact that the medicines have a particular property. The level-1 child node is a child node, and the level-2 child node is a child node of the level-1 child node, and the level-3 child node is a child node of the level-2 child node and so on. The level-1 parent node is a parent node, the level-2 parent node is a parent node of the level-1 parent node, and the level-3 parent node is a parent of the level-2 parent node, and so on.

In this embodiment, a sub-graph formed by some nodes and borders in the medical knowledge graph is labeled to form a training set. Therefore, the representation network and the determination network may be trained using the sub-graph. Afterwards, the properties of medicines corresponding to other medicine nodes in the medical knowledge graph may be predicted using the trained representation network and the trained determination network.

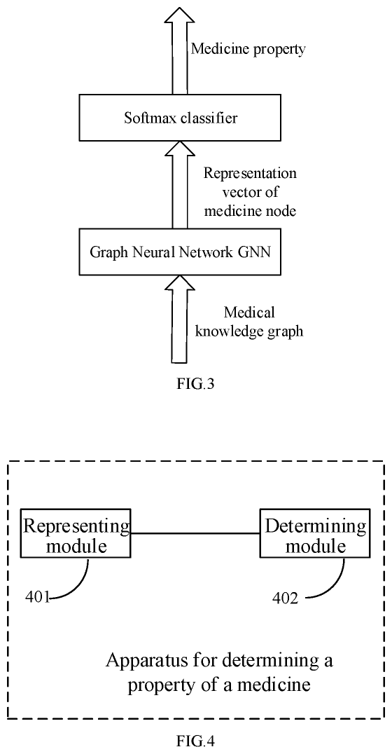

FIG. 3 illustrates a method of determining a property of a medicine according to an embodiment of the present disclosure. As can be seen from the drawing, with a graph neural network (GNN) as the representation network and a Softmax classifier as the determination network, the medical knowledge graph is input into the graph neural network, the graph neural network inputs a representation vector of a medicine node into the Softmax classifier, and the Softmax classifier outputs a property of the medicine, for example, whether the medicine is anti-inflammatory and the like.

Some embodiments of the present disclosure provide an apparatus for determining a property of a medicine. FIG. 4 illustrates a structural schematic diagram of the apparatus. The apparatus includes the following modules.

A representing module 401 is configured to input a medical knowledge graph into a pre-trained representation network, such that the representation network outputs a representation vector of at least one node of the medical knowledge graph.

A determining module 402 is configured to input a representation vector of a medicine node in the at least one node into a pre-trained determination network, such that the determination network outputs a property of a medicine corresponding to the medicine node.

In some embodiments of the present disclosure, the representing module is specifically configured to:
for each of the at least one node, obtain an initial vector by performing initial representation for the node; and
obtain and output a representation vector of the node by performing update on the initial vector at least once.

In some embodiments of the present disclosure, when used to perform the update on the initial vector at least once, the representing module is specifically configured to:

Perform the update on the initial vector at least once by using one or more parent nodes and/or one or more child nodes of the node, where the one or more parent node each indicate a node pointing to the node, and the one or more child nodes each indicate a node pointed to by the node.

In some embodiments of the present disclosure, when used to perform the update on the initial vector at least once by using the one or more parent nodes and/or one or more child nodes of the node, the representing module is specifically configured to:
update the vector based on a following formula:

$${h}^{t + 1} ( {e}_{i} ) = {\sum }_{{e}_{k} ∈ Np ( {e}_{i} )} σ ( {W}_{p} \times {e}_{i} + {W}_{ph} \times {h}^{t} ( {e}_{k} ) ) + {\sum }_{{e}_{j} ∈ Nc ( {e}_{i} )} σ ( {W}_{c} \times {e}_{i} + {W}_{ch} \times {h}^{t} ( {e}_{j} ) )$$

where eᵢ represents an i-th node in N nodes of the medical knowledge graph, and i=1,.. . . . . , N, represents an activation function, Np(eᵢ) represents a set of parent nodes of eᵢ, Nc(eᵢ) represents a set of child nodes of h^{t+1}(eᵢ) represents a vector obtained by performing update on the initial vector of eᵢ for t+1 times, h^{t}(eₖ) represents a vector obtained by performing update on the initial vector of eₖ for t times, h^{t}(eⱼ) represents a vector obtained by performing update on the initial vector of eⱼ for t times, where t is an integer equal to or greater than 1, and Wₚ, Wₚₕ, W_{c}, W_{ch} are network parameters of the representation network.

In some embodiments of the present disclosure, when used to obtain and output the representation vector of the node by performing the update on the initial vector at least once, the representing module is specifically configured to:

in response to that a time number of the update reaches a preset time number threshold, and/or, in response to that vectors after and before update are same, determine the vector obtained after the update as the representation vector of the node.

In some embodiments of the present disclosure, the determining module is specifically configured to:

based on a following formula, determine a probability that the medicine corresponding to the medicine node has the property:

$$p ( y = 1 \vert {e}_{i} ) = \frac{1}{1 + {e}^{- θ \times {h}^{n} ( {e}_{i} )}}$$

where tin (eᵢ) indicates a representation vector of medicine eᵢ and θ indicates a weight vector.

In some embodiments of the present disclosure, the apparatus further includes a querying module configured to:
store the probability that the medicine has the property;
receive medicine query information, where the medicine query information carries a medicine name and a property name;
according to the medicine query information and the stored probability that the medicine has the property, output a probability that a medicine corresponding to the medicine name has a property corresponding to the property name.

In some embodiments of the present disclosure, the apparatus further includes a training module, configured to:
train the representation network and/or the determination network by using a plurality of nodes in a training set, where one or more medicine nodes in the plurality of nodes each are labeled with one or more true properties of a corresponding medicine.

In some embodiments of the present disclosure, the training module is specifically configured to:
input each node in the training set into the representation network, such that the representation network outputs a representation vector of the node;
input a representation vector of a medicine node in the training set into the determination network, such that the determination network outputs a property of a medicine corresponding to the medicine node;
according to the output property of the medicine corresponding to the medicine node and the true property of the medicine corresponding to the medicine node, determine a network loss value; and
based on the network loss value, adjust one or more network parameters of the representation network and/or the determination network.

In some embodiments of the present disclosure, the apparatus further includes a training set preparing module, configured to:
mark labels for a plurality of medicine nodes of the medical knowledge graph, where the labels represent true properties of medicines corresponding to the medicine nodes;
determine a sub-graph formed by the plurality of medicine nodes and at least level-1 child node and parent node of each of the plurality of medicine nodes as the training set.

In some embodiments of the present disclosure, the apparatus further includes a representation network configured to:
add a border between the property node and the corresponding medicine node.

In some embodiments of the present disclosure, the medical knowledge graph includes one or more medicine nodes, one or more disease nodes and one or more category nodes.

In some embodiments of the present disclosure, the property of the medicine includes anti-inflammation and non-anti-inflammation.

Some embodiments of the present disclosure provide a system for determining a property of a medicine. FIG. 5 is a structural schematic diagram of the system. The system includes the following modules.

A representation network 501 is configured to: receive a medical knowledge graph, and output a representation vector of at least one node of the medical knowledge graph.

A determination network 502 is configured to: receive a representation vector of a medicine node in the at least one node, and output a property of a medicine corresponding to the medicine node.

The specific manners in which various modules and networks in the apparatus and system of the above embodiments perform operations are already described in details in the method embodiments of the third aspect, and will not be repeated herein.

Some embodiments of the present disclosure provide a system for providing medicine information. The system includes an inputting unit, a processor and a displaying unit.

The inputting unit is configured to receive medicine query information of a user.

The processor is electrically connected with the inputting unit and configured to determine a property of a medicine based on any method of determining a property of a medicine according to the present disclosure.

The displaying unit is electrically connected with the processor and configured to display the property of the medicine.

The system for providing medicine information according to the embodiments of the present disclosure is specifically a separate terminal device, and the terminal device may be an electronic device having strong computing power, such as a desktop computer, a laptop computer or two-in-one computer.

The system for providing medicine information according to the embodiments of the present disclosure includes a cloud device and a terminal device which are in communication. The cloud device may be an electronic device having strong computing power such as a single server, or a server cluster or a distributed server, and has a processor used to execute the steps of the method of determining a property of a medicine as mentioned above. The terminal device may be an electronic device having a weak computing power, such as smart phone or tablet computer, and have an inputting unit, a processor and a displaying unit.

In some embodiments, a probability that each medicine has the specific property is output or stored, for example, stored in a terminal device or server.

In some embodiments, when a user inputs a name of a medicine, a stored probability that the medicine has the specific property may be queried, and the probability that the medicine has the specific property may be output. For example, after the user inputs a name of a to-be-queried medicine through a terminal, a probability, which is stored in the terminal or the server, that the medicine has one or more specific properties may be retrieved, and the probability that the medicine has one or more specific properties may be output. For example, the user may input a name of a specific medicine and a specific property of the medicine to be queried at the same time, and the probability that the specific medicine has the specific property is output.

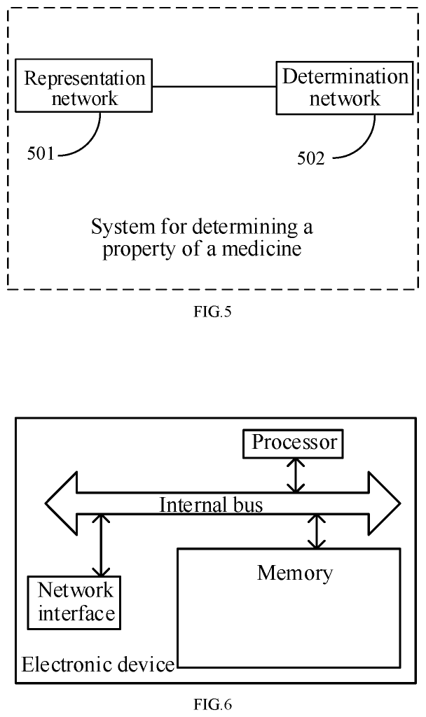

As shown in FIG. 6, some embodiments of the present disclosure provide an electronic device, including a memory and a processor. The memory is configured to store computer instructions executable on the processor, and the processor is configured to determine a property of a medicine based on the method of the first aspect when executing the computer instructions.

Some embodiments of the present disclosure provide a computer readable storage medium storing computer programs thereon, where the programs are executed by a processor to implement the method of the first aspect.

Each component embodiment of the present disclosure may be implemented in the form of hardware or a software module run in one or more processors or implemented by their combination.

It should be understood that although various steps in the flowchart of the accompanying drawings are displayed in a sequence as indicated by an arrow, these steps are not necessarily performed in the sequence indicated by the arrow. Unless otherwise clearly stated in the present disclosure, these steps are not limited to any strict sequence and may be performed in another sequence. Furthermore, at least part of the steps in the flowchart of the accompanying drawings may include a plurality of sub-steps or a plurality of stages. These sub-steps or stages are not necessarily completed at a same moment but may be performed at different moments. These sub-steps or stages are also not necessarily performed in sequence but may be performed in turns or alternately with at least part of other steps or the sub-steps or stages of other steps.

In the present disclosure, the terms “first” and “second” are used for the purpose of description only and shall not be understood as indicating or implying a relative importance. The term “plurality” refers to two or more unless otherwise clearly defined.

Other implementations of the present disclosure will be apparent to those skilled in the art from consideration of the specification and practice of the present disclosure herein. The present disclosure is intended to cover any variations, uses, modification or adaptations of the present disclosure that follow the general principles thereof and include common knowledge or conventional technical means in the related art that are not disclosed in the present disclosure. The specification and examples are considered as exemplary only, with a true scope and spirit of the present disclosure being indicated by the following claims.

It is to be understood that the present disclosure is not limited to the precise structure described above and shown in the accompanying drawings, and that various modifications and changes may be made without departing from the scope thereof. The scope of the present disclosure is limited only by the appended claims.

1. A method of determining a property of a medicine, comprising:
inputting a medical knowledge graph into a pre-trained representation network, such that the representation network outputs a representation vector of at least one node of the medical knowledge graph; and
inputting a representation vector of a medicine node in the at least one node into a pre-trained determination network, such that the determination network outputs a property of a medicine corresponding to the medicine node.

2. The method of claim 1, wherein the representation network outputs the representation vector of the at least one node of the medical knowledge graph, comprising:
for each of the at least one node, obtaining an initial vector by performing initial representation for the node; and
obtaining and outputting a representation vector of the node by performing update on the initial vector at least once.

3. The method of claim 2, wherein performing the update on the initial vector at least once comprises:
performing the update on the initial vector at least once by using one or more parent nodes and/or one or more child nodes of the node, wherein the one or more parent node each indicate a node pointing to the node, and the one or more child nodes each indicate a node pointed to by the node.

4. The method of claim 3, wherein performing the update on the initial vector at least once by using the one or more parent nodes and/or one or more child nodes of the node comprises:
updating the initial vector based on a following formula:
$${h}^{t + 1} ( {e}_{i} ) = {\sum }_{{e}_{k} ∈ Np ( {e}_{i} )} σ ( {W}_{p} \times {e}_{i} + {W}_{ph} \times {h}^{t} ( {e}_{k} ) ) + {\sum }_{{e}_{j} ∈ Nc ( {e}_{i} )} σ ( {W}_{c} \times {e}_{i} + {W}_{ch} \times {h}^{t} ( {e}_{j} ) )$$
wherein eᵢ represents an i-th node in N nodes of the medical knowledge graph, and i=1, . . . , N, represents an activation function, Np(eᵢ) represents a set of parent nodes of eᵢ, Nc(eᵢ) represents a set of child nodes of eᵢ, h^{t+1}(eᵢ) represents a vector obtained by performing update on the initial vector of eᵢ for t+1 times, h^{t}(eₖ) represents a vector obtained by performing update on the initial vector of eₖ for t times, h^{t}(eⱼ) represents a vector obtained by performing update on the initial vector of eⱼ for t times, where t is an integer equal to or greater than 1, and Wₚ, Wₚₕ, W_{c}, W_{ch} are network parameters of the representation network.

5. The method of claim 2, wherein obtaining the representation vector of the node by performing the update on the initial vector at least once comprises:
in response to that a time number of the update reaches a preset time number threshold, and/or, in response to that vectors after and before update are same, determining the vector obtained after the update as the representation vector of the node.

6. The method of claim 1, wherein the determination network outputs the property of the medicine corresponding to the medicine node, comprising:
based on a following formula, determining a probability that the medicine corresponding to the medicine node has the property:
$$p ( y = 1 \vert {e}_{i} ) = \frac{1}{1 + {e}^{- θ \times {h}^{n} ( {e}_{i} )}}$$
wherein hⁿ(eᵢ) indicates a representation vector of medicine eᵢ and θ indicates a weight vector.

7. The method of claim 6, further comprising:
storing the probability that the medicine has the property;
receiving medicine query information, wherein the medicine query information carries a medicine name and a property name;
according to the medicine query information and the stored probability that the medicine has the property, outputting a probability that a medicine corresponding to the medicine name has a property corresponding to the property name.

8. The method of claim 1, further comprising:
training the representation network and/or the determination network by using a plurality of nodes in a training set, wherein one or more medicine nodes in the plurality of nodes each are labeled with one or more true properties of a corresponding medicine.

9. The method of claim 8, wherein training the representation network and/or the determination network by using the plurality of nodes in the training set comprises:
inputting each node in the training set into the representation network, such that the representation network outputs a representation vector of the node;
inputting a representation vector of a medicine node in the training set into the determination network, such that the determination network outputs a property of a medicine corresponding to the medicine node;
according to the output property of the medicine corresponding to the medicine node and the true property of the medicine corresponding to the medicine node, determining a network loss value; and
based on the network loss value, adjusting one or more network parameters of the representation network and/or the determination network.

10. The method of claim 8, further comprising:
marking labels for a plurality of medicine nodes of the medical knowledge graph, wherein the labels represent true properties of medicines corresponding to the medicine nodes;
determining a sub-graph formed by the plurality of medicine nodes and at least level-1 child node and parent node of each of the plurality of medicine nodes as the training set.

11. The method of claim 1, further comprising:
updating the medical knowledge graph according to the property of the medicine output by the determination network, and adding a corresponding property attribute for the medicine node.

12. The method of claim 1, wherein the medical knowledge graph comprises one or more medicine nodes, one or more disease nodes and one or more category nodes.

13. The method of claim 1, wherein the property of the medicine comprises anti-inflammation and non-anti-inflammation.

14. (canceled)

15. A system for determining a property of a medicine, comprising:
a representation network, configured to receive a medical knowledge graph, and output a representation vector of at least one node of the medical knowledge graph; and
a determination network, configured to receive a representation vector of a medicine node in the at least one node and output a property of a medicine corresponding to the medicine node.

16. A system for providing medicine information, comprising:
an inputting unit, configured to receive medicine query information of a user;
a processor, electrically connected with the inputting unit and configured to determine a property of a medicine based on the method of determining a property of a medicine according to claim 1; and
a displaying unit, electrically connected with the processor and configured to display the property of the medicine.

17. An electronic device, comprising a memory and a processor,
wherein the memory is configured to store computer instructions executable on the processor, and the processor is configured to when executing the computer instructions, determine a property of a medicine according to operations comprising:
inputting a medical knowledge graph into a pre-trained representation network, such that the representation network outputs a representation vector of at least one node of the medical knowledge graph; and
inputting a representation vector of a medicine node in the at least one node into a pre-trained determination network, such that the determination network outputs a property of a medicine corresponding to the medicine node.

18. A non-transitory computer readable storage medium storing computer programs thereon, wherein the programs are executed by a processor to implement the method according to claim 1.

19. The electronic device of claim 17, wherein the representation network outputs the representation vector of the at least one node of the medical knowledge graph, comprising:
for each of the at least one node, obtaining an initial vector by performing initial representation for the node; and
obtaining and outputting a representation vector of the node by performing update on the initial vector at least once.

20. The electronic device of claim 19, wherein performing the update on the initial vector at least once comprises:
performing the update on the initial vector at least once by using one or more parent nodes and/or one or more child nodes of the node, wherein the one or more parent node each indicate a node pointing to the node, and the one or more child nodes each indicate a node pointed to by the node.

21. The electronic device of claim 20, wherein performing the update on the initial vector at least once by using the one or more parent nodes and/or one or more child nodes of the node comprises:
updating the initial vector based on a following formula:
$${h}^{t + 1} ( {e}_{i} ) = {\sum }_{{e}_{k} ∈ Np ( {e}_{i} )} σ ( {W}_{p} \times {e}_{i} + {W}_{ph} \times {h}^{t} ( {e}_{k} ) ) + {\sum }_{{e}_{j} ∈ Nc ( {e}_{i} )} σ ( {W}_{c} \times {e}_{i} + {W}_{ch} \times {h}^{t} ( {e}_{j} ) )$$
wherein eᵢ represents an i-th node in N nodes of the medical knowledge graph, and i=1, . . . , N, represents an activation function, Np(eᵢ) represents a set of parent nodes of eᵢ, Nc(eᵢ) represents a set of child nodes of eᵢ, h^{t+1}(eᵢ) represents a vector obtained by performing update on the initial vector of eᵢ for t+1 times, h^{t}(eₖ) represents a vector obtained by performing update on the initial vector of eₖ for t times, h^{t}(eⱼ) represents a vector obtained by performing update on the initial vector of eⱼ for t times, where t is an integer equal to or greater than 1, and Wₚ, W_{Ph}, W_{c}, W_{ch} are network parameters of the representation network.
